(19) United States
(12) Patent Application Publication
(10) Pub. No.: US 2015/0250635 A1
(43) Pub. Date: Sep. 10, 2015

(54) Adjustable Collar

(71) Applicant: Aspen Medical Partners, LLC, Irvine, CA (US)

(72) Inventors: Geoffrey Garth, Long Beach, CA (US); Albert Romo, Lakewood, CA (US); Jozsef Horvath, Fullerton, CA (US)

(21) Appl. No.: 14/638,519

(22) Filed: Mar. 4, 2015

Related U.S. Application Data
(60) Provisional application No. 61/947,676, filed on Mar. 4, 2014.

(51) Int. Cl. A61F 5/055

(52) U.S. Cl. CPC A61F 5/055; A61F 2250/0004; A61F 2250/001

(57) ABSTRACT

Adjustable braces providing improved levels of adjustability and ease of use are provided. Two components of an adjustable brace can be pivotally fastened to one another at two ends, and define a size-adjustable opening therebetween. The two components can additionally or alternatively be coupled to one another via an adjustment mechanism that allows for an adjustment and secured positioning of one component relative to the other via a simple manipulation of a single adjustment piece.

This application claims priority to U.S. provisional patent application no. 61/947676 filed Mar. 4, 2014. This and all other extrinsic materials discussed herein are incorporated by reference in their entirety.

### FIELD OF THE INVENTION

The field of the invention is orthosis, including adjustable collars.

### BACKGROUND

The following description includes information that may be useful in understanding the present invention. It is not an admission that any of the information provided herein is prior art or relevant to the presently claimed invention, or that any publication specifically or implicitly referenced is prior art.

Cervical collars are often used to maintain a spine in neutral alignment by supporting a user's chin at a particular position. Cervical collars can be supplied for temporary or long-term use, and emergency personnel typically have cervical collars readily available for use on patients having traumatic head or neck injuries. Because potential users of collars are sized and proportioned differently, and storage of bulky collars can be burdensome, many have attempted to provide one-size-fits-most adjustable collars that can be used by different users having different or varying heights, weights and proportions.

For example, U.S. Pat. No. 5,795,315 to Traut teaches a generally flat neck encircling band that can be sized to a standard size prior to being worn, and a mandible support that couples to the band via slots, sliders, locking holes and locking tabs such that the mandible support can be positioned at one of a plurality of vertical positions relative to the band.

This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Unfortunately, the Traut collar apparently cannot be adjusted when worn, requires separate adjustment and locking members, requires the separate adjustment and locking members to be located at opposing extremeties of the mandible support, and suffers from various other disadvantages.

Thus, there is still a need for improved adjustable collars.

### SUMMARY OF THE INVENTION

The inventive subject matter provides apparatus, systems and methods in which two components of a brace can be coupled to one another in a manner that allows for an adjustment and secured positioning of one component relative to the other via a simple manipulation of a single adjustment piece.

In some aspects, a curved chin support is pivotally attached to a curved collar body on right and left sides forming a size-adjustable tracheal opening therebetween. The chin support includes a chin cup and a centrally located single adjustment piece coupled with the chin cup. The adjustment piece includes at least one attachment point and at least two end portions that can couple with notched members of the collar body, the two end portions each including rod-like members. One exemplary adjustment piece has an upside-down U-shape, where the free end portions of the “U” are considered the two free ends of the adjustment piece.

The collar body includes left and right notched members, each including a plurality of vertically arranged notches. The left notched member is located on the collar body such that each of its notches are positioned, sized and dimensioned to separately receive the left free end's rod-like member. The right notched member is located on the collar body such that each of its notches are positioned, sized and dimensioned to separately receive the right free end's rod-like member.

Preferably, the adjustment piece is biased such that its rod-like members are each seated in a notch of the notched members. In such embodiments, the adjustment piece can simply be moved away from a pair of notches in which its rod-like members are seated, up or down to align with a desired pair of different notches, and released to be seated therein. One or more locking mechanisms could be included, but are not required because the adjustment piece is biased towards an engaged position.

It should be appreciated that a rod-like member could have any suitable cross-sectional shape (e.g., circle, oval, square, rectangle, triangle, pentagon, hexaton, diamond or irregular) and does not need to be a circular bar.

In other aspects, a rear support could be provided for fastening to the front portion of the brace (chin support and collar body). The rear support could include right and left side fasteners that are complementary to left and right side fasteners located on an outer surface of the front portion of the brace. Preferably the fasteners allow for adjustment of a circumference of the brace to accommodate necks of different sizes (e.g., hook and loop fasteners, snap fasteners).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a front perspective view of a brace of the inventive subject matter.

FIG. 1B is a left-side perspective view of the brace of FIG. 1A.

FIG. 2A is a side perspective view of the adjustment mechanism of the brace of FIGS. 1A-1B.

FIG. 2B illustrates a rear view of the U-shaped Adjustment Piece of FIG. 2A.

FIG. 3A is a side view of the brace of FIG. 1A-2B in a first configuration.

FIG. 3B is a side view of the brace of FIG. 1A-2B in a second configuration.

FIG. 3C is a side view of the brace of FIG. 1A-2B in a third configuration.

FIG. 3D is a side view of the brace of FIG. 1A-2B in a fourth configuration.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The inventive subject matter provides apparatus, systems and methods in which two components of a brace can be coupled to one another in a manner that allows for an adjustment and secured positioning of one component relative to the other via a simple manipulation of a single adjustment piece. Viewed from another perspective, the braces described herein allow the left and right sides of one component of a brace to be simultaneously adjusted relative to another component via a manipulation of a single centrally-located adjustment piece.

As used herein, the term “centrally-located” should be interpreted broadly to include an object whose midline is located at the midline of a second object or anywhere within 15% of the midline in either direction (e.g., up/down or left/right) relative to the entire length of the second object.

While the discussion herein is generally directed to cervical collars, one should appreciate that the adjustment mechanisms provided herein could be used with any suitable orthotic device having at least two components that adjustably (e.g., pivotally) couple.

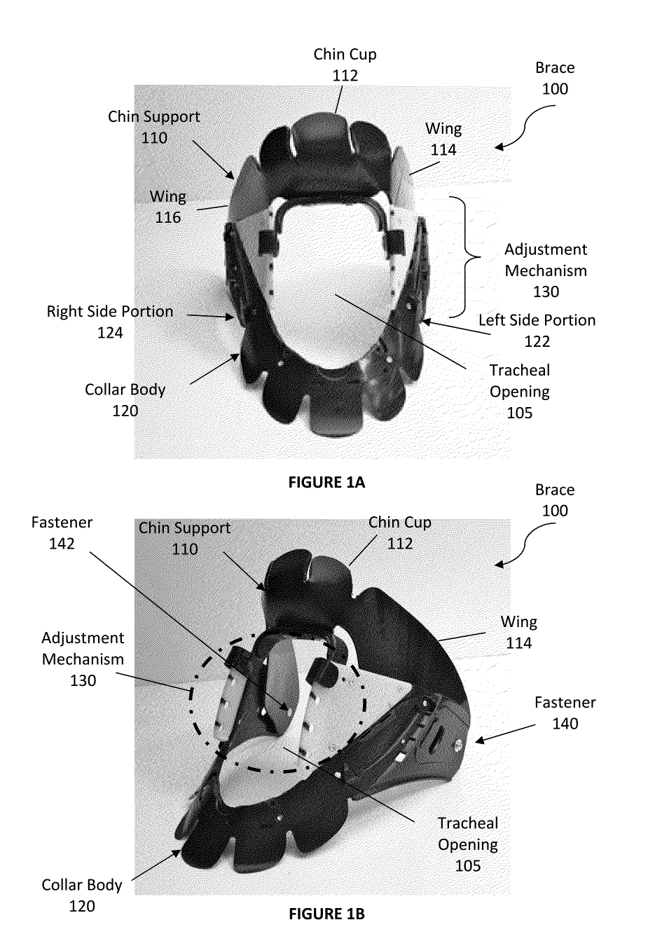

An exemplary adjustable collar of the inventive subject matter is shown in FIGS. 1A-1B. FIG. 1A illustrates a front perspective view of brace 100, and FIG. 1B illustrates a left-side perspective views of brace 100. Brace 100 includes a chin support 110 and collar body 120, which are pivotally coupled to one another via fasteners (e.g., 140 and 142) and define a size-adjustable tracheal opening 105 that provides access for airway management and other procedures.

As used herein, the term “size-adjustable tracheal opening” refers to an opening of a brace that provides an access point to a wearer's trachea (e.g., for a tracheotomy) when the brace is worn, and is adjustable in size along at least one of a diameter, a length and a width.

Chin support 110 includes a chin cup 112 and laterally extending left and right side wings 114 and 116 located on opposite sides of the chin cup 112. The chin cup and laterally extending wings could be made of a single piece of material, or could comprise separate pieces of material that are coupled to one another. Collar body 120 includes a front portion (shown here as being toothed) and left and right side portions 122, 124, respectively. Similarly to the chin support, the front and side portions could be made of a single piece of material, or could comprise separate pieces of material that are coupled with one another. The two components (chin support 110 and collar body 120) are pivotally fastened to one another via a first fastener 140 that couples wing 114 and left side portion 122, and a second fastener 142 that couples wing 116 and right side portion 124.

While chin supports and collar bodies of the inventive subject matter are preferably made at least in part of sturdy and stiff materials such as molded thermoplastics (e.g., polyethylene) or thermoset plastics such that each can permanently maintain a generally curved shape that contours to the neck of a wearer, it should be appreciated that the chin support and collar body could each be made of any suitable material or materials. In some preferred braces, the inner surface of the chin support and collar body are coupled with padding material(s) via hook and loop or other fastener(s) such that the inner surfaces of the chin support and collar body are lined partially or entirely with a padding material to prevent or reduce discomfort to a wearer.

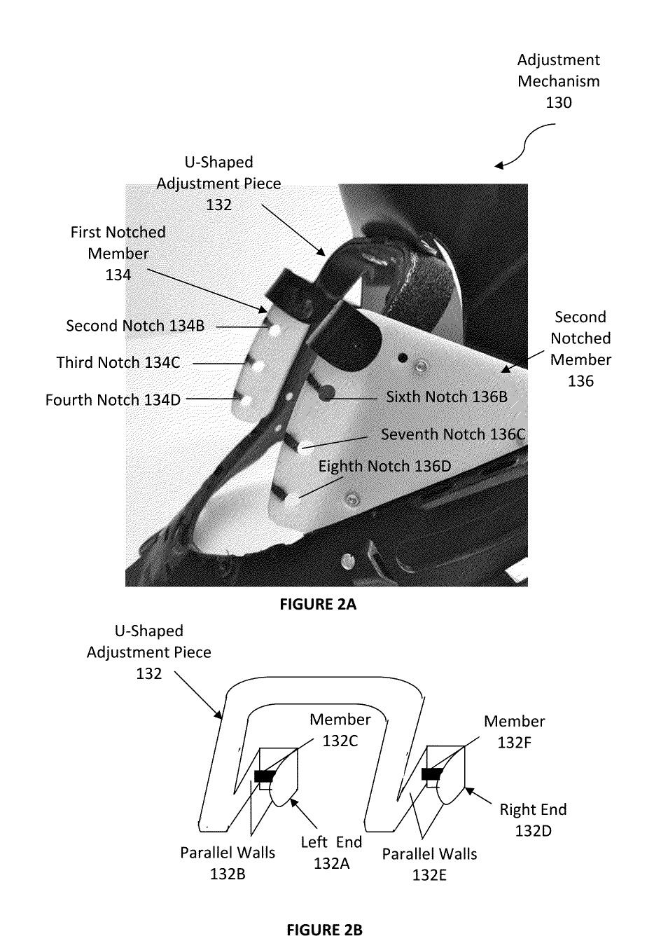

Brace 100 also includes an adjustment mechanism 130, which is shown in detail in FIGS. 2A-2B. Adjustment mechanism 130 advantageously allows a user (such as a wearer or a medical or emergency professional) to adjust a vertical position of chin support 110 relative to the collar body 120 via a single-handed manipulation of a centrally located adjustment piece. Viewed from another perspective, adjustment mechanism 130 allows a user to adjust a size of a tracheal opening formed by the chin support and collar body via a single-handed manipulation of a centrally located adjustment piece.

As illustrated, adjustment mechanism 130 includes a U-shaped adjustment piece 132 that is coupled with chin cup 112. Adjustment piece 132 could be fixedly or movably coupled with the chin cup, for example, via one or more screws, hinges, or other fasteners. While adjustment pieces are generally described herein as being U-shaped, it should be appreciated that adjustment piece could comprise any suitable shape that includes at least one attachment point and at least two end portions that can couple with notched members of the collar body.

Where the adjustment piece 132 is fixedly attached to chin piece 112, it is contemplated that the adjustment piece 132 could be movable from an unengaged position to an engaged position via the flexibility of the chin cup 112. For example, the chin cup could be toothed such that the chin cup can more easily be manipulated. Where the adjustment piece 132 is movably attached to chin piece 112 (e.g., pivotally attached via a hinge and spring biasing mechanism), the adjustment piece 132 could be movable from an unengaged position to an engaged position without flexing or deforming of the chin cup 112.

U-shaped adjustment piece 132 includes a left end 132A and a right end 132D, each of which comprise a pair of parallel walls, 132B and 132E, respectively, and a member 132C and 132F, respectively, that extend between the parallel walls. Additionally, adjustment mechanism 130 includes a first notched member 134 and a second notched member 136 disposed on right and left sides of the collar body 120, respectively. While members 132C and 132F are shown to be disposed between parallel walls of the U-shaped adjustment piece, it is contemplated that the members could instead be located to project from the left and right ends 132A and 132D, respectively.

As used herein, the term “notched member” should be interpreted broadly to include members having two or more recesses, indentations or incisions along an edge, as well as members having one or more openings (through-holes) there-through. Similarly, the term “notch” should be interpreted broadly to include a recess, indentation or incision, as well as an opening. As one non-limiting example, a notched member could comprise a notched rail or slot including a vertical portion that guides a protrusion, projection or rod-like member to a plurality of substantially perpendicular portions (notches) where the protrusion, projection or rod-like member could rest.

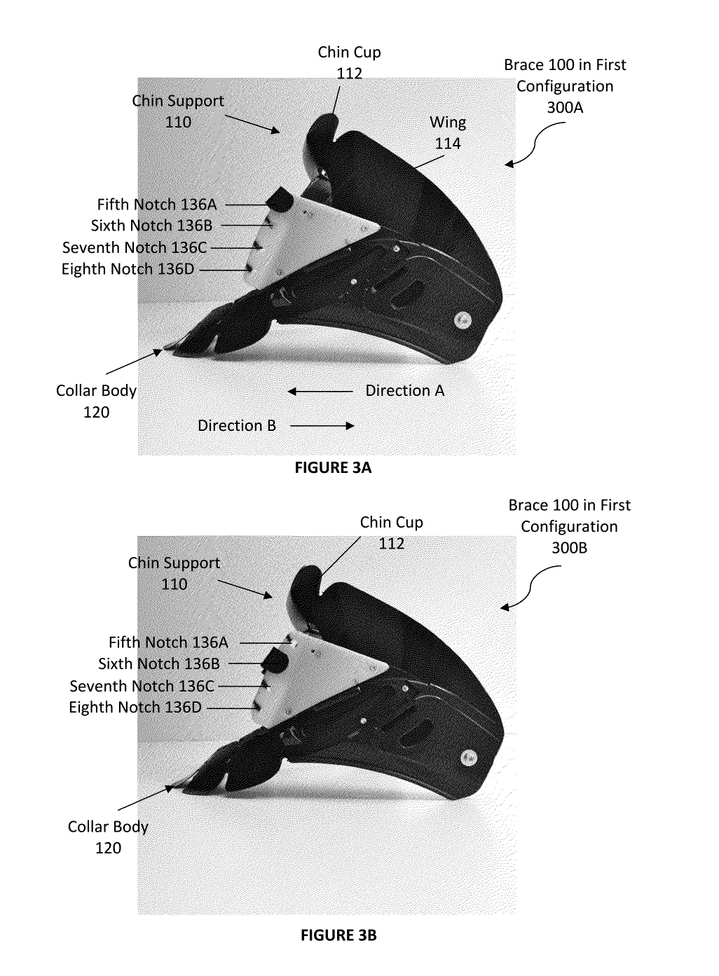

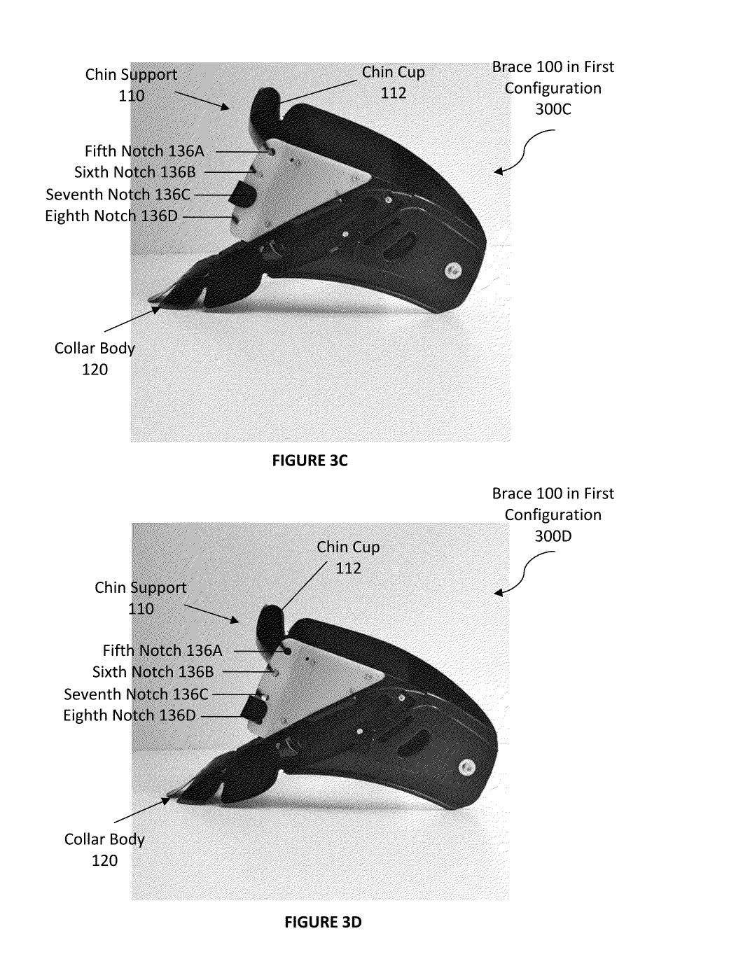

First notched member 134 includes a plurality of notches including first notch 134A (see FIGS. 3A-3D), second notch 134B, third notch 134C and fourth notch 134D. Second notched member 136 includes a plurality of notches including fifth notch 136A (see FIGS. 3A-3D), sixth notch 136B, seventh notch 137C and eighth notch 136D. Preferably each of the notches of the first and second notched members are horizontally aligned with at least one other notch (e.g., 134A-136A, 134B-136B, 134C-136C, 134D-136D), and sized and dimensioned to separately receive member 132C or 132F.

While brace 100 has an adjustment piece 132 that is disposed on the chin support 110 (e.g., coupled with chin piece 112) and notched members 134 and 136 disposed on the collar body 120, it should be appreciated that in some embodiments the adjustment piece could be disposed on the collar body while the notched members could be disposed on sides of the chin support such that the notches vertically align with the adjustment piece's left and right ends.

As illustrated in FIGS. 3A-3D, brace 100 can be adjusted to be in one of at least four configurations. It should be appreciated that a brace of the inventive subject matter could include any suitable number of notches (e.g., 2, 4, 6, 8, 10 or 12) based on a number of different configurations suitable for the brace. For example, where a brace is an adjustable collar and 6 different configurations (lengths between chin cup and collar body) are desired, each of two notched members could comprise 6 notches, totaling 12 notches.

In FIG. 3A, brace 100 is in configuration 300A, which provides the greatest vertical distance between chin cup 112 of chin support 110 and a fixed point (e.g., bottommost central point) of collar body 120. In configuration 300A, first notch 134A and fifth notch 136A are horizontally aligned, and left end member 132C and right end member 132F are similarly horizontally aligned. Additionally, left end member 132C is disposed partially or fully within fifth notch 136A and right end member 132F is disposed partially or fully within first notch 134A. As U-shaped adjustment piece 132 is biased in Direction B and notches 134A and 136A can optionally be slanted down and inwards, no locking mechanism is required to keep brace 100 in configuration 300A (or 300B, 300C, 300D), even while brace 100 is being worn.

In FIG. 3B, brace 100 is in configuration 300B, which provides for a shorter vertical distance between chin cup 112 and the fixed point of collar body 120 than configuration 300A.

In configuration 300B, left end member 132C is disposed partially or fully within sixth notch 136B and right end member 132F is disposed partially or fully within second notch 134B, which is horizontally aligned with sixth notch 136B. Brace 100 could be adjusted from configuration 300A to 300B via a simple manipulation of adjustment piece 132 in Direction A, and release of adjustment piece 132 in Direction B once members 132C and 132F are aligned with notches 136B and 134B. Viewed from another perspective, manipulation of adjustment piece 132 simultaneously moves members 132C and 132F from a first pair of notches (134A and 136A) to a second pair of notches (134B and 136B).

In FIG. 3C, brace 100 is in configuration 300C, which provides for a shorter vertical distance between chin cup 112 and the fixed point of collar body 120 than configurations 300A and 300B. In configuration 300C, left end member 132C is disposed partially or fully within sixth notch 136C and right end member 132F is disposed partially or fully within second notch 134C, which is horizontally aligned with sixth notch 136C.

In FIG. 3D, brace 100 is in configuration 300D, which provides for a shorter vertical distance between chin cup 112 and the fixed point of collar body 120 than configurations 300A, 300B and 300C. In configuration 300D, left end member 132C is disposed partially or fully within sixth notch 136D and right end member 132F is disposed partially or fully within second notch 134D, which is horizontally aligned with sixth notch 136D.

It should be apparent to those skilled in the art that many more modifications besides those already described are possible without departing from the inventive concepts herein. The inventive subject matter, therefore, is not to be restricted except in the spirit of the appended claims. Some other contemplated embodiments of the inventive subject matter include, but are not limited to those described in co-owned U.S. patent application Ser. No. 14/638366, incorporated herein by reference.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. An adjustable collar comprising:
a chin support coupled to a collar body via an adjustment mechanism;
wherein the adjustment mechanism comprises:
first and second notched members located on the collar body, wherein the first notched member comprises a first plurality of notches including a first upper notch and a first lower notch, and wherein the second notched member comprises a second plurality of notches including a second upper notch and a second lower notch; and
a U-shaped adjustment piece located below the chin support and movable between an unengaged position and an engaged position, wherein a first end of the U-shaped adjustment piece comprises a first member sized and dimensioned to separately engage with each of the first upper notch and the first lower notch when in the engaged position, and wherein a second end of the U-shaped adjustment piece comprises a second member sized and dimensioned to separately engage with each of the second upper notch and the second lower notch when in the engaged position; and
wherein the U-shaped adjustment piece is biased towards the engaged position; and
wherein a manipulation of the U-shaped adjustment piece simultaneously moves the first and second members from the first and second lower notches, respectively, to the first and second upper notches, respectively.

2. The adjustable collar of claim 1, wherein the chin support comprises a chin cup and a pair of laterally extending wings.

3. The adjustable collar of claim 1, wherein the chin support comprises a chin cup, and wherein the U-shaped adjustment piece is fixedly attached to the chin cup.

4. The adjustable collar of claim 1, wherein the chin support comprises a chin cup, and wherein the U-shaped adjustment piece is movably attached to the chin cup.

5. The adjustable collar of claim 1, wherein the first member of the U-shaped adjustment piece extends between two substantially parallel walls of the first end, and wherein the second member of the U-shaped adjustment piece extends between two substantially parallel walls of the second end.

6. The adjustable collar of claim 1, wherein the chin support comprises a chin cup and first and second laterally extending wings, wherein the collar body comprises a first end and a second end, wherein the first laterally extending wing is pivotally coupled with the first end of the collar body, and wherein the second laterally extending wing is pivotally coupled with the second end of the collar body.

7. The adjustable collar of claim 1, wherein the collar body comprises a front portion, a left side portion and a right side portion, and wherein the first and second notched members are fixedly attached to the left and right side portions, respectively.

8. The adjustable collar of claim 1, further comprising a padding material coupled to an inner surface of at least one of the chin support and the collar body.

9. An adjustable collar comprising:
a chin support coupled to a collar body via an adjustment mechanism;
wherein the adjustment mechanism comprises:
a first pair of horizontally aligned notches disposed on left and right inner edges of the collar body;
a second pair of horizontally aligned notches disposed on the left and right inner edge of the collar body at a vertical position below the first pair of notches; and
an adjustment piece coupled to the chin support and comprising first and second ends, wherein the first and second ends comprise first and second members sized and dimensioned to separately engage with the first pair of notches and the second pair of notches; and
wherein the adjustment piece is movable relative to the chin support from an engaged position to an unengaged position, wherein the first and second members are sized and dimensioned to at least partially rest in first and second notches of the first pair of notches, and separately in first and second notches of the second pair of notches when the adjustment piece is in the engaged position, and wherein no portion of the first and second members are disposed within any notch of the first and second pair of notches when the adjustment piece is in the unengaged position.

10. The adjustable collar of claim 9, wherein the chin support comprises a chin cup and a pair of laterally extending wings.

11. The adjustable collar of claim 9, wherein the chin support comprises a chin cup, and wherein the adjustment piece is fixedly attached to the chin cup.

12. The adjustable collar of claim 9, wherein the chin support comprises a chin cup, and wherein the adjustment piece is movably attached to the chin cup.

13. The adjustable collar of claim 9, wherein the first member of the adjustment piece extends between two substantially parallel walls of the first end, and wherein the second member of the adjustment piece extends between two substantially parallel walls of the second end.

14. The adjustable collar of claim 9, wherein the chin support comprises a chin cup and first and second laterally extending wings, wherein the collar body comprises a first end and a second end, wherein the first laterally extending wing is pivotally coupled with the first end of the collar body, and wherein the second laterally extending wing is pivotally coupled with the second end of the collar body.

15. The adjustable collar of claim 9, wherein the collar body comprises a front portion, a left side portion and a right side portion, and wherein the first and second pairs of horizontally aligned notches are disposed on first and second notched members that are fixedly attached to the left and right side portions, respectively.

16. The adjustable collar of claim 9, further comprising a padding material coupled to an inner surface of at least one of the chin support and the collar body.

17. The adjustable collar of claim 9, wherein the adjustment piece comprises a U-shape.
